(19) United States
(12) Patent Application Publication
(10) Pub. No.: US 2022/0324795 A1
(43) Pub. Date: Oct. 13, 2022

(54) PROCESS FOR PRODUCING 4,4'-DICHLORODIPHENYL SULFONE

(71) Applicant: BASF SE, Ludwigshafen am Rhein (DE)

(72) Inventors: Jessica Nadine HAMANN, Ludwigshafen am Rhein (DE); Stefan BLEI, Ludwigshafen am Rhein (DE); Oliver BEY, Ludwigshafen am Rhein (DE)

(21) Appl. No.: 17/635,142

(22) PCT Filed: Aug. 20, 2020
(86) PCT No.: PCT/EP2020/073365

§ 371 Date: Feb. 14, 2022

(30) Foreign Application Priority Data
Aug. 27, 2019 (EP) 19193681.4

(51) Int. Cl. C07C 315/02; C07C 317/14

(52) U.S. Cl. CPC C07C 315/02; C07C 317/14

(57) ABSTRACT

The invention relates to a process for producing 4,4′-dichlorodiphenyl sulfone comprising reacting a solution comprising 4,4′-dichlorodiphenyl sulfoxide and at least one linear C₆-C₁₀ carboxylic acid as solvent with an oxidizing agent to obtain a crude reaction product comprising 4,4′-dichlorodiphenyl sulfone, wherein the concentration of water in the reaction mixture is kept below 5 wt %, the process comprising: (a) adding 0.9 to 1.05 mol oxidizing agent per mol 4,4′-dichlorodiphenyl sulfoxide uniformly distributed to the solution at a temperature in the range from 80 to 105° C. over a period from 1.5 to 5 h in a first step to obtain the reaction mixture; (b) agitating the reaction mixture after completion of the first step at the temperature of the first step for 5 to 30 min without adding oxidizing agent; (c) adding 0.05 to 0.2 mol oxidizing agent per mol 4,4′-dichlorodiphenyl sulfoxide to the reaction mixture at a temperature in the range from 80 to 105° C. over a period of less than 40 min in a second step; (d) agitating the reaction mixture after completion of the second step at the temperature of the second step for 10 to 30 min without adding oxidizing agent, (e) heating the reaction mixture to a temperature in the range from 95 to 110° C. and hold this temperature for 10 to 90 min to obtain a crude reaction product comprising 4,4′-dichlorodiphenyl sulfone.

The invention relates to a process for producing 4,4′-dichlorodiphenyl sulfone by oxidizing 4,4′-dichlorodiphenyl sulfoxide with an oxidizing agent in a carboxylic acid as solvent.

4,4′-dichlorodiphenyl sulfone (in the following DCDPS) is used for example as a monomer for preparing polymers like polyether sulfone or polysulfone or as an intermediate of pharmaceuticals, dyes and pesticides.

Several methods for obtaining DCDPS are known. DCDPS for example is produced by oxidation of 4,4′-dichlorodiphenyl sulfoxide (in the following also termed as DCDPSO). The latter can be obtained for instance by a Friedel-Crafts reaction of thionyl chloride and chlorobenzene as starting materials in the presence of a catalyst, for example aluminum chloride.

A process for producing an organic sulfone by oxidation of the respective sulfoxide in the presence of at least one peroxide is disclosed in WO-A 2018/007481. The reaction thereby is carried out in a carboxylic acid as solvent, the carboxylic acid being liquid at 40° C. and having a miscibility gap with water at 40° C. and atmospheric pressure.

It is an object of the present invention to provide a reliable and energy-efficient process for producing 4,4′-dichlorodiphenyl sulfone with a reduced amount of impurities, particularly remainders of 4,4′-dichlorodiphenyl sulfoxide which did not convert into DCDPS.

This object is achieved by a process for producing 4,4′-dichlorodiphenyl sulfone comprising reacting a solution comprising 4,4′-dichlorodiphenyl sulfoxide and at least one linear C₆-C₁₀ carboxylic acid as solvent with an oxidizing agent to obtain a crude reaction product comprising 4,4′-dichlorodiphenyl sulfone, wherein the concentration of water in the reaction mixture is kept below 5 wt %, the process comprising:
(a) adding 0.9 to 1.05 mol oxidizing agent per mol 4,4′-dichlorodiphenyl sulfoxide uniformly distributed to the solution at a temperature in the range from 80 to 105° C. over a period from 1.5 to 5 h in a first step to obtain the reaction mixture;
(b) agitating the reaction mixture after completion of the first step at the temperature of the first step for 5 to 30 min without adding oxidizing agent;
(c) adding 0.05 to 0.2 mol oxidizing agent per mol 4,4′-dichlorodiphenyl sulfoxide to the reaction mixture at a temperature in the range from 80 to 105° C. over a period of less than 40 min in a second step;
(d) agitating the reaction mixture after completion of the second step at the temperature of the second step for 10 to 30 min without adding oxidizing agent,
(e) heating the reaction mixture to a temperature in the range from 95 to 110° C. and hold this temperature for 10 to 90 min to obtain a crude reaction product comprising 4,4′-dichlorodiphenyl sulfone.

Surprisingly it has been shown that by keeping the concentration of water below 5 wt % in the reaction mixture, the conversion of 4,4′-dichlorodiphenyl sulfoxide forming DCDPS can be improved. Further, keeping the concentration of water below 5 wt % allows using a linear C₆-C₁₀ carboxylic acid which is only slightly health hazardous and which has a good biodegradability.

Another advantage of using the linear C₆-C₁₀ carboxylic acid is that the linear C₆-C₁₀ carboxylic acid shows a good separability from water at low temperatures which allows separation of the linear C₆-C₁₀ carboxylic acid without damaging the product and which further allows recycling the linear C₆-C₁₀ carboxylic acid as solvent into the oxidation process.

Particularly, by this process it is possible to obtain a crude reaction product comprising DCDPS which contains less than 1000 ppm DCDPSO based on the sum of DCDPS and DCDPSO.

In the process for producing DCDPS a solution comprising DCDPSO and at least one linear C₆-C₁₀ carboxylic acid (in the following termed as carboxylic acid) is provided. In this solution, the carboxylic acid serves as solvent. Preferably, the ratio of DCDPSO to carboxylic acid is in a range from 1:2 to 1:6, particularly in a range from 1:2.5 to 1:3.5. Such a ratio of DCDPSO to carboxylic acid is usually sufficient to completely solve the DCDPSO in the carboxylic acid at the reaction temperature and to achieve an almost full conversion of the DCDPSO forming DCDPS and further to use as little carboxylic acid as possible. The solution comprising DCDPSO and carboxylic acid preferably is heated to a temperature in the range from 70 to 110° C., more preferred to a temperature in the range from 80 to 100° C. and particularly in the range from 85 to 95° C., for example 86, 87, 88, 89, 90, 91, 92, 93, 94° C., before adding the oxidizing agent.

To provide the solution, it is possible to feed DCDPSO and the carboxylic acid separately into a reactor and to mix the DCDPSO and the carboxylic acid in the reactor. Alternatively, it is also possible to mix the DCDPSO and the carboxylic acid in a separate mixing unit to obtain the solution and to feed the solution into the reactor. In a further alternative, DCDPSO and a part of the carboxylic acid are fed into the reactor as a mixture and the rest of the carboxylic acid is fed directly into the reactor and the solution is obtained by mixing the mixture of DCDPSO and part of the carboxylic acid and the rest of the carboxylic acid in the reactor.

The linear C₆-C₁₀ carboxylic acid can be only one carboxylic acid or a mixture of at least two different carboxylic acids. Preferably the carboxylic acid is at least one aliphatic carboxylic acid. Preferably the aliphatic carboxylic acid is aliphatic monocarboxylic acid. Thus, the at least one carboxylic acid may be n-hexanoic acid, n-heptanoic acid, n-octanoic acid, n-nonanoic acid or n-decanoic acid or a mixture of one or more of said acids. Particularly preferably, however, the carboxylic acid is n-hexanoic acid or n-heptanoic acid.

Heating of the solution comprising DCDPSO and the carboxylic acid can be carried out in the reactor in which the reaction for obtaining the crude reaction product takes place or in any other apparatus before being fed into the reactor. Particularly preferably, the solution comprising DCDPSO and the carboxylic acid is heated to the respective temperature before being fed into the reactor. Heating of the solution for example can be carried out in a heat exchanger through which the solution flows before being fed into the reactor or more preferred in a buffer container in which the solution is stored before being fed into the reactor. If such a buffer container is used, the buffer container also may serve as mixing unit for mixing the DCDPSO and the carboxylic acid to obtain the solution.

A heat exchanger for example can be used when the process is operated continuously. Heating of the solution in a buffer container can be carried out in a continuously operated process as well as in a batchwise operated process. If a heat exchanger is used for heating the solution, any suitable heat exchanger can be used, for example a shell and tube heat exchanger, a plate heat exchanger, a spiral tube heat exchanger, or any other heat exchanger known to a skilled person. The heat exchanger thereby can be operated in counter current flow, co-current flow or cross flow.

Besides heating by using a heating fluid which usually is used in a heat exchanger or for heating in a double jacket or heating coil, also electrical heating or induction heating can be used for heating the solution.

If the solution is heated in the buffer container, any suitable container which allows heating of the contents in the container can be used. Suitable containers for example are equipped with a double jacket or a heating coil. If the buffer container additionally is used for mixing the DCDPSO and the carboxylic acid, the buffer container further comprises a mixing unit, for example a stirrer.

For carrying out the reaction, the solution preferably is provided in a reactor. This reactor can be any reactor which allows mixing and reacting of the components fed into the reactor. A suitable reactor for example is a stirred tank reactor or a reactor with forced circulation, particularly a reactor with external circulation and a nozzle to feed the circulating liquid. If a stirred tank reactor is used, any stirrer can be used. Suitable stirrers for example are axially conveying stirrers like oblique blade agitators or cross-arm stirrers or radially conveying agitators like flat blade agitators. The stirrer may have at least 2 blades, more preferred at least 4 blades. Particularly preferred is a stirrer having 4 to 8 blades, for example 6 blades. For reasons of process stability and process reliability, it is preferred that the reactor is a stirred tank reactor with an axially conveying stirrer.

For controlling the temperature in the reactor, it is further preferred to use a reactor with heat exchange equipment, for example a double jacket or a heating coil. This allows additional heating or heat dissipation during the reaction and keep the temperature constant or in a predefined temperature range at which the reaction is carried out. Preferably, the reaction temperature is kept in a range from 70 to 110° C., more preferred from 80 to 100° C. and particularly in a range from 85 to 95° C., for example 86, 87, 88, 89 90, 91, 92, 93, 94° C.

To obtain DCDPS, the DCDPSO in the solution comprising DCDPSO and carboxylic acid is oxidized by an oxidizing agent. Therefore, the oxidizing agent is added to the solution to obtain a reaction mixture. From the reaction mixture the crude reaction product comprising DCDPS can be obtained.

The oxidizing agent used for oxidizing DCDPSO for obtaining DCDPS preferably is at least one peroxide. The at least one peroxide may be at least one peracid, for example one or a mixture of two or more, such as three or more peracids. Preferably, the process disclosed herein is carried out in the presence of one or two, particularly in the presence of one peracid. The at least one peracid may be a C₁ to C₁₀ peracid, which may be unsubstituted or substituted, e.g. by linear or branched C₁ to C₅ alkyl or halogen, such as fluorine. Examples thereof are peracetic acid, performic acid, perpropionic acid, percaprionic acid, pervaleric acid or pertrifluoroacetic acid. Particularly preferably the at least one peracid is a C₆ to C₁₀ peracid, for example 2-ethylhexanoic peracid. If the at least one peracid is soluble in water, it is advantageous to add the at least one peracid as aqueous solution. Further, if the at least one peracid is not sufficiently soluble in water, it is advantageous that the at least one peracid is dissolved in the respective carboxylic acid. Most preferably, the at least one peracid is a linear C₆ to C₁₀ peracid which is generated in situ.

Particularly preferably, the peracid is generated in situ by using hydrogen peroxide (H₂O₂) as oxidizing agent. At least a part of the added H₂O₂ reacts with the carboxylic acid forming the peracid. The H₂O₂ preferably is added as an aqueous solution, for instance as 1 to 90 wt % solution, such as a 20, 30, 40, 50, 60, 70 or 80 wt % solution, preferably as 30 to 85 wt % solution, particularly as a 50 to 85 wt % solution, each being based on the total amount of the aqueous solution. Using a highly concentrated aqueous solution of H₂O₂, particularly a solution of 50 to 85 wt %, for example of 70 wt %, based on the total amount of the aqueous solution, may lead to a reduction of reaction time. It may also facilitate recycling of the at least one carboxylic acid.

To avoid accumulation of the oxidizing agent and to achieve a constant oxidation of the DCDPSO, it is preferred to add the oxidizing agent continuously with a controlled feed rate, for example with a feed rate from 0.002 to 0.01 mol per mol DCDPSO and minute. More preferred, the oxidizing agent is added with a feed rate from 0.003 to 0.008 mol per mol DCDPSO and minute and particularly with a feed rate from 0.004 to 0.007 mol per mol DCDPSO and minute.

The oxidizing agent can be added with a constant feed rate or with a varying feed rate. If the oxidizing agent is added with a varying feed rate, it is for example possible to reduce the feed rate with proceeding reaction within the above described range. The oxidizing agent is added in several steps with a stop of adding oxidizing agent between the steps. In each step during adding the oxidizing agent, the oxidizing agent can be added with a constant feed rate or a varying feed rate. Besides a decreasing feed rate with proceeding reaction, it is also possible to increase the feed rate or to switch between increasing and decreasing feed rates. If the feed rate is increased or decreased, the change in feed rate can be continuously or stepwise. Particularly preferably, the oxidizing agent is added in at least two steps wherein the feed rate in each step is constant.

If the oxidation of DCDPSO is carried out in at least two steps, for converting the DCDPSO into DCDPS, the DCDPSO is oxidized by adding the oxidizing agent in the first and second steps to the solution comprising DCDPSO and carboxylic acid.

In the first step 0.9 to 1.05 mol oxidizing agent per mol 4,4′-dichlorodiphenyl sulfoxide are added uniformly distributed to the solution at a temperature in the range from 70 to 110° C. over a period from 1.5 to 5 h. By adding the oxidizing agent over such a period an accumulation of the oxidizing agent can be avoided.

“Uniformly distributed” in this context means that the oxidizing agent can be added either continuously at a constant feed rate or at periodically changing feed rates. Besides continuous periodically changing feed rates, periodically changing feed rates also comprise discontinuously changing periodical feed rates for example feed rates where oxidizing agent is added for a defined time, then no oxidizing agent is added for a defined time and this adding and not adding is repeated until the complete amount of oxidizing agent for the first step is added. The period in which the oxidizing agent is added, is in a range from 1.5 to 5 h, more preferred in a range from 2 to 4 h and particularly in a range from 2.5 to 3.5 h. By adding the oxidizing agent uniformly distributed over such a period, it can be avoided that oxidizing agent accumulates in the reaction mixture which may result in an explosive mixture. Additionally, by adding the oxidizing agent over such a period, the process can be scaled up in an easy way as this allows also in an upscaled process to dissipate the heat from the process. On the other hand, by such an amount decomposition of the hydrogen peroxide is avoided and thus the amount of hydrogen peroxide used in the process can be minimized.

The temperature at which the first step is carried out is in the range from 70 to 110° C., preferably in the range from 85 to 100° C. and particularly in the range from 90 to 95° C. In this temperature range, a high reaction velocity can be achieved at high solubility of the DCDPSO in the carboxylic acid. This allows to minimize the amount of carboxylic acid and by this a controlled reaction can be achieved.

After the addition of the oxidizing agent in the first step is completed, the reaction mixture is agitated at the temperature of the first step for 5 to 30 min without adding oxidizing agent. By agitating the reaction mixture after completion of adding the oxidizing agent, oxidizing agent and DCDPSO which did not yet react are brought into contact to continue the reaction forming DCDPS for reducing the amount of DCDPSO remaining as impurity in the reaction mixture.

To further reduce the amount of DCDPSO in the reaction mixture, after completing of agitating without adding oxidizing agent, 0.05 to 0.2 mol oxidizing agent per DCDPSO, preferably 0.06 to 0.15 mol oxidizing agent per mol DCDPSO, and particularly 0.08 to 0.1 mol oxidizing agent per mol DCDPSO are added to the reaction mixture in the second step.

In the second step, the oxidizing agent preferably is added in a period from 1 to 40 min, more preferred in a period from 5 to 25 min and particularly in a period from 8 to 15 min. The addition of the oxidizing agent in the second step may take place in the same way as in the first step. Further, it is also possible to add the entire oxidizing agent of the second step at once.

The temperature of the second step is in the range from 80 to 110° C., more preferred in the range from 85 to 100° C. and particularly in the range from 93 to 98° C. It further is preferred that the temperature in the second step is from 3 to 10° C. higher than the temperature in the first step. More preferred the temperature in the second step is 4 to 8° C. higher than the temperature in the first step and particularly preferably, the temperature in the second step is 5 to 7° C. higher than the temperature in the first step. By the higher temperature in the second step, it is possible to achieve a higher reaction velocity.

After addition of the oxidizing agent in the second step, the reaction mixture is agitated at the temperature of the second step for 10 to 20 min to continue the oxidation reaction of DCDPSO forming DCDPS.

To complete the oxidation reaction, after agitating at the temperature of the second step without adding oxidizing agent, the reaction mixture is heated to a temperature in the range from 95 to 110° C., more preferred in the range from 95 to 105° C. and particularly in the range from 98 to 103° C. and held at this temperature for 10 to 90 min, more preferred from 10 to 60 min and particularly from 10 to 30 min.

In the oxidizing process, particularly when using H₂O₂ as oxidizing agent, water is formed. Further, water may be added with the oxidizing agent. According to the invention, the concentration of the water in the reaction mixture is kept below 5 wt %, more preferred below 3 wt % and particularly below 2 wt %. By using aqueous hydrogen peroxide with a concentration of 70 to 85 wt % the concentration of water during the oxidization reaction is kept low. It even may be possible to keep the concentration of water in the reaction mixture during the oxidization reaction below 5 wt % without removing water by using aqueous hydrogen peroxide with a concentration of 70 to 85 wt %.

Additionally or alternatively, it may be necessary to remove water from the process for keeping the concentration of water in the reaction mixture below 5 wt %. To remove the water from the process, it is for example possible to strip water from the reaction mixture. Stripping thereby preferably is carried out by using an inert gas as stripping medium. If the concentration of water in the reaction mixture remains below 5 wt % when using aqueous hydrogen peroxide with a concentration of 70 to 85 wt % it is not necessary to additionally strip water. However, even in this case it is possible to strip water to further reduce the concentration.

Suitable inert gases which can be used for stripping the water are non-oxidizing gases and are preferably nitrogen, carbon dioxide, noble gases like argon or any mixture of these gases. Particularly preferably, the inert gas is nitrogen.

The amount of inert gas used for stripping the water preferably is in the range from 0 to 2 Nm³/h/kg, more preferably in the range from 0.2 to 1.5 Nm³/h/kg and particularly in the range from 0.3 to 1 Nm³/h/kg. The gas rate in Nm³/h/kg can be determined according to DIN 1343, January 1990 as relative gas flow. Stripping of water with the inert gas may take place during the whole process or during at least one part of the process. If water is stripped at more than one part of the process, between the parts stripping of water is interrupted. The interruption of stripping water is independent of the mode in which the oxidizing agent is added. For example, it is possible to add the oxidizing agent without any interruption and to strip the water with interruptions or to add the oxidizing agent in at least two steps and to strip the water continuously. Further it is also possible, to strip water only during the addition of oxidizing agent. Particularly preferably, the water is stripped by continuously bubbling an inert gas into the reaction mixture.

To avoid the formation of areas with different compositions in the reactor which may lead to different conversion rates of DCDPSO and thus to different yield and amounts of impurities, it is preferred to homogenize the reaction mixture during the first step and the second step. Homogenization of the reaction mixture can be performed by any method known to a skilled person, for example by agitating the reaction mixture. To agitate the reaction mixture, it is preferred to stir the reaction mixture. For stirring, any suitable stirrer can be used. Suitable stirrers for example are axially conveying stirrers like oblique blade agitators or cross-arm stirrers or radially conveying agitators like flat blade agitators. The stirrer may have at least 2 blades, more preferred at least 4 blades. Particularly preferred is a stirrer having 4 to 8 blades, for example 6 blades. For reasons of process stability and process reliability, it is preferred that the reactor is a stirred tank reactor with an axially conveying stirrer.

The temperature of the reaction mixture during the process can be set for example by providing a pipe inside the reactor through which a tempering medium can flow. Under the aspect of ease of reactor maintenance and/or uniformity of heating, preferably, the reactor comprises a double jacket through which the tempering medium can flow. Besides the pipe inside the reactor or the double jacket the tempering of the reactor can be performed in each manner known to a skilled person, for example by withdrawing a stream of the reaction mixture from the reactor, passing the stream through a heat exchanger in which the stream is tempered and recycle the tempered stream back into the reactor.

To support the oxidation reaction, it is further advantageous to additionally add at least one acidic catalyst to the reaction mixture. The acidic catalyst may be at least one, such as one or more, such as a mixture of two or three additional acids. An additional acid in this context is an acid which is not the carboxylic acid which serves as solvent. The additional acid may be an inorganic or organic acid, with the additional acid preferably being an at least one strong acid. Preferably, the strong acid has a pKₐ value from −9 to 3, for instance −7 to 3 in water. A person skilled in the art appreciates that such acid dissociation constant values, Kₐ, can be for instance found in a compilation such as in IUPAC, Compendium of Chemical Terminology, 2^{nd} ed. “Gold Book”, Version 2.3.3, Feb. 24, 2014, page 23. The person skilled in the art appreciates that such pKₐ values relate to the negative logarithm value of the Kₐ value. it is more preferred that the at least one strong acid has a negative pKₐ value, such as from −9 to −1 or −7 to −1 in water.

Examples for inorganic acids being the at least one strong acid are nitric acid, hydrochloric acid, hydrobromic acid, perchloric acid, and/or sulfuric acid. Particularly preferably, one strong inorganic acid is used, in particular sulfuric acid. While it may be possible to use the at least one strong inorganic acid as aqueous solution, it is preferred that the at least one inorganic acid is used neat. Suitable strong organic acids for example are organic sulfonic acids, whereby it is possible that at least one aliphatic or at least one aromatic sulfonic acid or a mixture thereof is used. Examples for the at least one strong organic acid are para-toluene sulfonic acid, methane sulfonic acid or trifluormethane sulfonic acid. Particularly preferably the strong organic acid is methane sulfonic acid. Besides using either at least one inorganic strong acid or at least one organic strong acid, it is also possible to use a mixture of at least one inorganic strong acid and at least one organic strong acid as acidic catalyst. Such a mixture for example may comprise sulfuric acid and methane sulfonic acid.

The acidic catalyst preferably is added in catalytic amounts. Thus, the amount of acidic catalyst used may be in the range from 0.001 to 0.3 mol per mol DCDPSO, for example in the range from 0.1 to 0.3 mol per mol DCDPSO, more preferred in the range from 0.15 to 0.25 mol per mol DCDPSO. However, it is particularly preferred to employ the acidic catalyst in an amount of less than 0.1 mol per mol DCDPSO, such as in an amount from 0.001 to 0.08 mol per mol DCDPSO, for example from 0.001 to 0.03 mol per mol DCDPSO. Particularly preferably, the acidic catalyst is used in an amount from 0.005 to 0.03 mol per mol DCDPSO.

The inventive process for obtaining DCDPS can be carried out as a batch process, as a semi continuous process or as a continuous process. Preferably, the process is carried out batchwise. The process can be carried out at atmospheric pressure or at a pressure which is below or above atmospheric pressure, for example in a range from 10 to 900 mbar(abs). Preferably, the process is carried out at a pressure in a range from 200 to 800 mbar(abs) and particularly in a range from 350 to 700 mbar(abs), such as 400, 500 or 600 mbar(abs). Surprisingly, the reduced pressure has the additional advantage that the total conversion of DCDPS can be increased and thus a very low content of remaining DCDPS in the product can be achieved. The process can be carried out under ambient atmosphere or inert atmosphere. If the process is carried out under inert atmosphere, it is preferred to purge the reactor with an inert gas before feeding the DCDPSO and the carboxylic acid. If the process is carried out under an inert atmosphere and the water formed during the oxidation reaction is stripped with an inert gas, it is further preferred that the inert gas used for providing the inert atmosphere and the inert gas which is used for stripping the water is the same. It is a further advantage of using an inert atmosphere that the partial pressure of the components in the process, particularly the partial pressure of water is reduced.

By the inventive process, a reaction mixture is obtained which comprises 4,4′-dichlorodiphenyl sulfone solved in the at least one carboxylic acid. To achieve DCDPS from the reaction mixture, the reaction mixture may be further worked up. By working up the reaction mixture, a crude reaction product comprising DCDPS and carboxylic acid are obtained. For separating the DCDPS from the carboxylic acid any process known to a skilled person can be used. Suitable processes for working up the crude reaction product for example are distillation or crystallization processes.

The carboxylic acid separated from the reaction mixture preferably is reused in the process as solvent and therefore recycled into the reaction.

The process described above can be carried out in only one apparatus or in more than one apparatus depending on the apparatus size and the amounts of compounds to be added. If more than one apparatus is used, the apparatuses can be operated simultaneously or—particularly in a batchwise operated process—at different time. This allows for example to carry out a process in one apparatus while at the same time another apparatus is maintained, for example cleaned. Further, it is possible after feeding the compounds in one apparatus to feed the components into a further apparatus while the process in the first apparatus still continues. However, it is also possible to add the components into all apparatus simultaneously and to carry out the processes in the apparatus also simultaneously.

### EXAMPLES

Example Reaction without Split H₂O₂ Dosage

1000.1 g of 4,4′-dichlorodiphenyl sulfoxide were dissolved in 3000 g n-heptanoic acid and heated to 90° C. 1.2 g sulfuric acid were added to the solution. Over a period of 3 h and 15 min 188 g H₂O₂ were added to the solution with a constant feed rate. During the reaction the temperature in the vessel was controlled to 90° C. by wall cooling, whereby the temperature in the reactor was determined to be 96 to 98° C. After completion of the H₂O₂ dosage the temperature of the thus obtained reaction mixture was raised to 98° C. The reaction mixture was stirred for 25 minutes at a temperature of 98° C. The reaction thereby was carried out at a pressure of 500 mbar (abs) and 12 NL/h nitrogen were passed through the reaction mixture for stripping water.

Subsequently, the reaction mixture was cooled to 20° C. by which the 4,4′-dichlorodiphenyl sulfone crystallized and a suspension formed comprising 4,4′-dichlorodiphenyl sulfone crystals and a mother liquor. The suspension was subjected to a filtration obtaining a filter cake comprising the 4,4′-dichlorodiphenyl crystals and 2999 g mother liquor as filtrate.

The resulting content of 4,4′-dichlorodiphenyl sulfoxide in the 4,4′-dichlorodiphenyl sulfone crystals was 1050 ppm (determined by gas chromatography).

The mother liquor obtained by the solid-liquid separation contained 3.15 g 4,4′-dichlorodiphenyl sulfoxide. Thus, the conversion rate of the 4,4′-dichlorodiphenyl sulfoxide was 99.68%.

Example Reaction with Split H₂O₂ Dosage

1111 g of 4,4′-dichlorodiphenyl sulfoxide were dissolved in 2900 g n-heptanoic acid and heated to 90° C. 7.2 g sulfuric acid were added to the solution. Over a period of 3 h and 5 min 197 g 70% H₂O₂ were added to the solution with a constant feed rate. During the reaction the temperature in the vessel was controlled to 90° C. by wall cooling, whereby the temperature in the reactor was determined to be 97 to 99° C. After finishing this step, the thus obtained reaction mixture was stirred for 15 minutes at a temperature of 97° C. Then, a second amount of 10 ml H₂O₂ was added within 10 minutes. After completing the H₂O₂ dosage the temperature of the reaction mixture was raised to 103° C. The reaction mixture was stirred for 20 minutes at this temperature. The reaction thereby was carried out at a pressure of 650 mbar (abs) and 10 NL/h nitrogen were passed through the reaction mixture for stripping water.

Subsequently, the reaction mixture was cooled to 20° C. by which the 4,4′-dichlorodiphenyl sulfone crystallized and a suspension formed comprising 4,4′-dichlorodiphenyl sulfone crystals and a mother liquor. The suspension was subjected to a filtration obtaining a filter cake comprising the 4,4′-dichlorodiphenyl crystals and 2900 g mother liquor as filtrate.

The resulting content of 4,4′-dichlorodiphenyl sulfoxide in the 4,4′-dichlorodiphenyl sulfone crystals was below the detection limit (determined by gas chromatography).

The mother liquor obtained by the solid-liquid separation contained 0.5807 g 4,4′-dichlorodiphenyl sulfoxide. Thus, the conversion rate of the 4,4′-dichlorodiphenyl sulfoxide was 99.95%.

1.-14. (canceled)

15. A process for producing 4,4′-dichlorodiphenyl sulfone comprising reacting a solution comprising 4,4′-dichlorodiphenyl sulfoxide and at least one linear C₆-C₁₀ carboxylic acid as solvent with an oxidizing agent to obtain a crude reaction product comprising 4,4′-dichlorodiphenyl sulfone, wherein the concentration of water in the reaction mixture is kept below 5 wt %, the process comprising:
(a) adding 0.9 to 1.05 mol oxidizing agent per mol 4,4′-dichlorodiphenyl sulfoxide uniformly distributed to the solution at a temperature in the range from 80 to 105° C. over a period from 1.5 to 5 h in a first step to obtain the reaction mixture;
(b) agitating the reaction mixture after completion of the first step at the temperature of the first step for 5 to 30 min without adding oxidizing agent;
(c) adding 0.05 to 0.2 mol oxidizing agent per mol 4,4′-dichlorodiphenyl sulfoxide to the reaction mixture at a temperature in the range from 80 to 105° C. over a period of less than 40 min in a second step;
(d) agitating the reaction mixture after completion of the second step at the temperature of the second step for 10 to 30 min without adding oxidizing agent,
(e) heating the reaction mixture to a temperature in the range from 95 to 110° C. and hold this temperature for 10 to 90 min to obtain a crude reaction product comprising 4,4′-dichlorodiphenyl sulfone.

16. The process according to claim 15, wherein for keeping the concentration of water below 5 wt % water is stripped from the reaction mixture.

17. The process according to claim 15, wherein the oxidizing agent is aqueous hydrogen peroxide with a concentration of 50 to 85 wt %.

18. The process according to claim 15, wherein the oxidizing agent is added continuously with a feed rate from 0.002 to 0.01 mol per mol 4,4′-dichlorodiphenyl sulfoxide and minute.

19. The process according to claim 15, wherein the temperature in the second step is 3 to 8° C. higher than the temperature in the first phase.

20. The process according to claim 15, wherein the reaction mixture is homogenized during the first step and the second step.

21. The process according to claim 15, wherein the reaction is carried out at a pressure in the range from 10 to 900 mbar(abs).

22. The process according to claim 15, wherein the solution is heated to a temperature in the range from 70 to 110° C. before adding the oxidizing agent.

23. The process according to claim 15, wherein the linear C₆-C₁₀ carboxylic acid is n-hexanoic acid and/or n-heptanoic acid.

24. The process according to claim 15, wherein an acidic catalyst is added to the reaction mixture.

25. The process according to claim 24, wherein the amount of acidic catalyst added to the reaction mixture is in the range from 0.001 to 0.3 mol per mol 4,4′-dichlorodiphenyl sulfoxide.

26. The process according to claim 15, wherein the reaction mixture is worked up to obtain a crude reaction product comprising 4,4′-dichlorodiphenyl sulfone and carboxylic acid.

27. The process according to claim 26, wherein the carboxylic acid is recycled into the reaction.

28. The process according to claim 15, wherein the process is carried out batchwise.
